(19) United States
(12) Patent Application Publication
(10) Pub. No.: US 2022/0383874 A1
(43) Pub. Date: Dec. 1, 2022

(54) DOCUMENTATION SYSTEM BASED ON DYNAMIC SEMANTIC TEMPLATES

(71) Applicant: 3M Innovative Properties Company, St. Paul, MN (US)

(72) Inventor: Detlef Koll, Pittsburgh, PA (US)

(21) Appl. No.: 17/333,713

(22) Filed: May 28, 2021

(51) Int. Cl. G10L 15/26; G16H 10/60; G06F 40/35; G10L 15/07; G16H 15/00; G06F 40/186

(52) U.S. Cl. CPC G10L 15/26; G16H 10/60; G06F 40/35; G10L 15/07; G16H 15/00; G06F 40/186

(57) ABSTRACT

A computer system and method transcribe a spoken dialogue, such as a dialogue between a physician and a patient, into a document, such as a clinical note. As the document is generated, if content is detected in the dialog which corresponds to a content template, the content template is inserted into the document. Fields in the content template may also be filled using information from the dialog and/or information external to the dialog.

### BACKGROUND

Various computer systems exist for generating transcripts of speech automatically or semi-automatically. Examples of such systems are those which generate clinical documents based on (live or recorded) dialogues between physicians and patients during healthcare encounters. One challenge is to implement systems which are capable of generating transcripts and other documents which are complete and comply with best practices.

### SUMMARY

A computer system and method transcribe a spoken dialogue, such as a dialogue between a physician and a patient, into a document, such as a clinical note. As the document is generated, if content is detected in the dialog which corresponds to a content template, the content template is inserted into the document. Fields in the content template may also be filled using information from the dialog and/or information external to the dialog.

Other features and advantages of various aspects and embodiments of the present invention will become apparent from the following description and from the claims.

### BRIEF DESCRIPTION OF THE DRAWINGS

FIG. 1 illustrates a system configured to identify and insert content templates into a document, according to embodiments of the present invention.

FIGS. 2A and 2B illustrate methods perform by the system of FIG. 1 according to embodiments of the present invention.

### DETAILED DESCRIPTION

Various computer systems exist for generating transcripts of speech automatically or semi-automatically. Examples of such systems are those which generate clinical documents based on (live or recorded) dialogues between physicians and patients during healthcare encounters. The information content of such dialogues is difficult to express discretely using ontologies. However, it can be possible to express a subset of the information content of a dialogue discretely, e.g., using an ontology. Embodiments of the present invention may identify one or more subsets of the information in a dialog, and store such information discretely, e.g., in a document, using an ontology or other discrete form. As a result, embodiments of the present invention may advantageously generate documents based on speech using less human effort and enable such documents to be searched and otherwise processed more efficiently than documents generated by prior art systems. Another advantage of embodiments of the present invention is that they are capable of encoding the meaning of a content template within the content template, thereby leading to lower error rates than prior art systems. Yet another advantage of embodiments of the present invention is that the content generated by embodiments of the present invention is more complete than content generated manually by physicians.

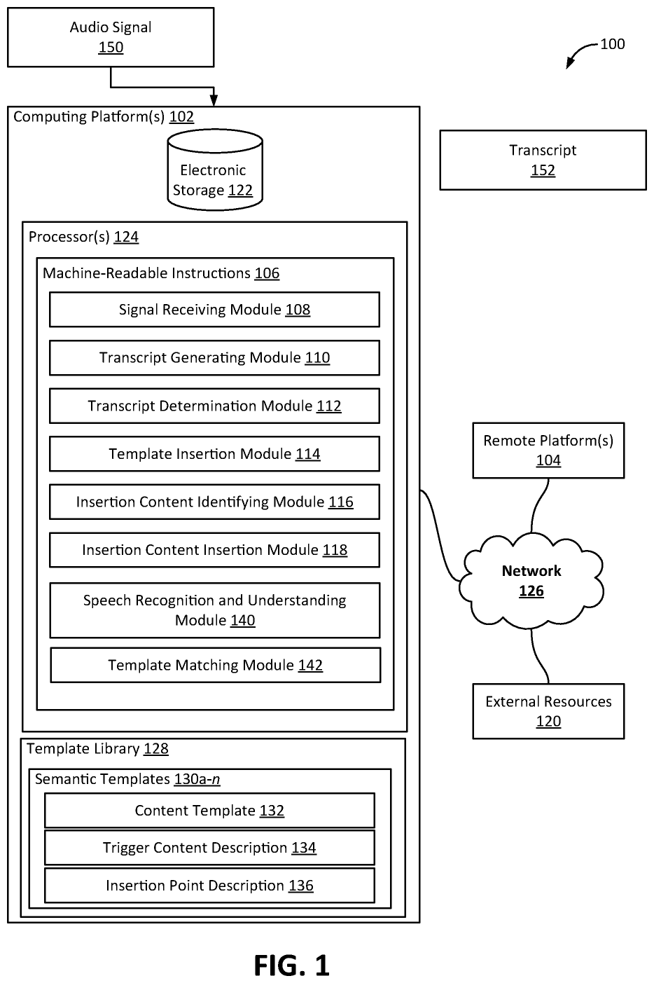

FIG. 1 illustrates a system 100 configured to identify and insert content templates into a document according to embodiments of the present invention. In some implementations, system 100 may include one or more computing platforms 102. Although the computing platform(s) 102 may include one or more computers, element 102 may be referred to herein in the singular as a “computing platform” for ease of explanation. The computing platform(s) 102 may include one or more computers, as that term is used herein. Computing platform(s) 102 may be configured to communicate with one or more remote platforms 104 over a network 126 according to a client/server architecture, a peer-to-peer architecture, and/or other architectures. Remote platform(s) 104 may be configured to communicate with other remote platforms via computing platform(s) 102 and/or according to a client/server architecture, a peer-to-peer architecture, and/or other architectures. Users may access system 100 via remote platform(s) 104. The network 126 may, for example, be the public Internet, a private intranet, or any other kinds of telecommunications network.

Computing platform(s) 102 may be configured by machine-readable instructions 106, which may be stored on one or more non-transitory computer-readable media. Machine-readable instructions 106 may include one or more instruction modules. The instruction modules may include computer program modules. The instruction modules may include one or more of signal receiving module 108, transcript generating module 110, transcript determination module 112, template insertion module 114, insertion content identifying module 116, insertion content insertion module 118, and/or other instruction modules.

The system 100 (e.g., the computing platform 102) may include a library 128 of semantic templates 130a-n, where n may be any number. Each of the semantic templates 130a-n includes a corresponding:
content template 132 with zero, one, or more fields to be filled with information captured from a dialogue;
semantic description 134 of content (also referred to herein as a “trigger content description”) which triggers the insertion of the corresponding content template 132; and
an insertion point description 136, which describes a location of an insertion point at which to insert the content template 132 into the transcript 152.

Although only a single content template 132 and corresponding semantic description of triggering content 134 is shown in FIG. 1 for ease of illustration, in practice each of the semantic templates 130a-n includes its own distinct content template 132 and semantic description 134. For example, a first one of the semantic templates 130a-n may include a first content template and corresponding first semantic description of triggering content, and a second one of the semantic templates 130a-n may include a second content template and corresponding second semantic description of triggering content. The first content template may differ from the second content template, and the first semantic description of triggering content may differ from the second semantic description of triggering content. The first semantic description of triggering content may describe content that triggers the insertion of the first content template, and the second semantic description of triggering content may describe content that triggers the insertion of the second content template.

Each content template 132 may take any of a variety of forms and represent any of a variety of information. For example, at a minimum, each content template 132 may contain some text. Different content templates may contain different text. In addition, some or all of the content templates 132 may include one or more fields to be filled with information captured from a dialog, as described in more detail below. Some of the content templates 132 may not include any such fields; such content templates 132 may, for example, include only text and not contain any fields.

Fields in the content templates may include data (e.g., information models) indicating which information is to be filled into those templates. For example, such a field may include data which specifies a concept, thereby indicating that data representing that concept is to be filled into that field. As a particular example, such a field may include an information model representing an “allergy” concept. Such a concept may have one or more parameters with corresponding values (e.g., allergen and treatment in the case of an allergy concept).

Each of the trigger content descriptions 134 may describe the corresponding triggering content in any of a variety of ways. For example, a particular one of the trigger content descriptions 134 may include a corresponding set of one or more coded clauses. Then, when the system 100 is generating a document based on a dialogue, if the system 100 detects one of those coded clauses in the dialogue, the system 100 may, in response to such detection, insert the content template corresponding to the particular one of the trigger content descriptions 134 into the document.

Techniques for performing such detection and insertion will be described in more detail below. As one example, the system may detect one or more particular keywords and, in response to that detection, insert the content template corresponding to the detected keyword(s) into the document. As another example, the system may detect that the dialogue includes content representing a particular topic and, in response to that detection, insert the content template corresponding to the detected concept into the document. As yet another example, the system may detect that the dialogue includes content representing a particular data element (e.g., a particular allergen) and, in response to that detection, insert the content template corresponding to the detected data element into the document.

Any element that is illustrated in FIG. 1 as being contained within the processor(s) 124 (such as the machine-readable instructions 106 and the library 128) may, additionally or alternatively, be contained within one or more non-transitory computer-readable media, such as the electronic storage 122 in the computing platform(s) 102.

The system 100 (e.g., the computing platform(s) 102) also include a speech recognition and understanding module 140, which may include, for example, both an automatic speech recognition (ASR) engine and a natural language understanding (NLU) engine. The ASR engine may, for example, be any of a variety of well-known ASR engines, such as MModal Fluency Direct or MModal Fluency Assistant. The NLU engine may, for example, be any of a variety of well-known NLU engines, such as 3M OneNLU. The speech recognition and understanding module 140 may include, for example, one or both of a speaker change detection module and a speaker identification module.

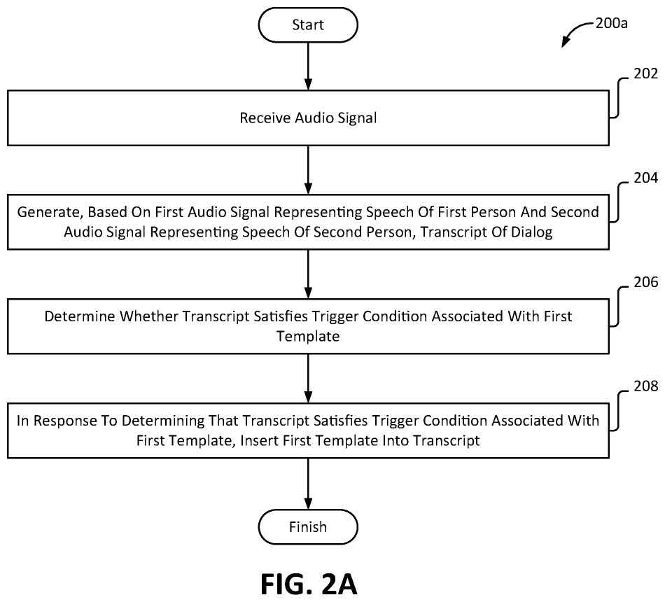

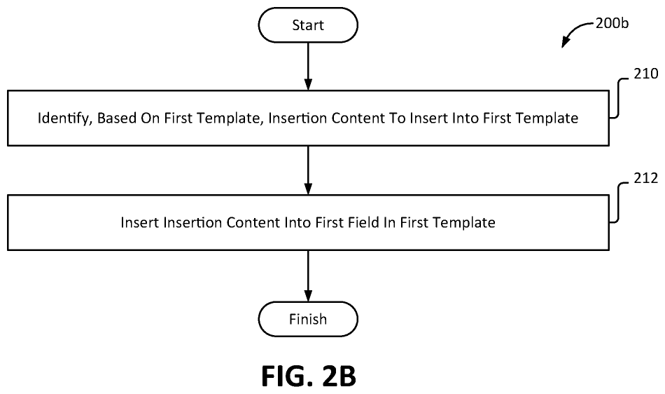

FIGS. 2A-2B illustrate methods 200a-b, respectively, performed by the system 100 of FIG. 1 in accordance with one or more implementations. The operations of methods 200a-b presented below are intended to be illustrative. In some implementations, methods 200a-b may be accomplished with one or more additional operations not described, and/or without one or more of the operations discussed. Additionally, the order in which the operations of methods 200a-b are illustrated in FIGS. 2A and/or 2B and described below is not intended to be limiting.

In some implementations, methods 200a-b may be implemented in one or more processing devices (e.g., the computing platform 102 and/or remote platform 104 of FIG. 1). The one or more processing devices may include one or more devices executing some or all of the operations of methods 200a-b in response to instructions stored electronically on an electronic storage medium. The one or more processing devices may include one or more devices configured through hardware, firmware, and/or software to be specifically designed for execution of one or more of the operations of method 200a-b.

Signal receiving module 108 may be configured to receive an audio signal 150 (FIG. 2A, operation 202). The audio signal 150 may represent speech of one or more people, such as speech including a dialogue between a first person and a second person (e.g., a physician and a patient). The audio signal 150 may include a first audio signal representing speech of the first person and a second audio signal representing speech of the second person.

The audio signal 150 may, for example, be a live audio signal, a recorded audio signal, or a combination thereof. For example, the system 100 may include one or more audio capture devices (e.g., microphones), which may capture speech of one or more people and generate, as output, the audio signal 150 representing that speech. In this case, the signal receiving module 108 may receive the audio signal 150 in real-time or substantially in real-time, as the audio signal 150 is being generated. Alternatively, for example, the speech of one or more people may be captured and recorded onto a computer-readable medium. The audio signal 150 may be such a recorded signal. In this case, some or all of the audio signal 150 may be received by the signal receiving module 108 after some or all of the audio signal 150 has been stored in the computer-readable medium.

Transcript generating module 110 may be configured to receive, as input, some or all of the audio signal 150 and to generate, based on the audio signal 150, a transcript 152 (FIG. 2A, operation 204). For example, although the transcript generating module 110 and the speech recognition and understanding module 140 are shown as distinct modules in FIG. 1, in practice those two modules may overlap or be implemented as a single module, or overlap in a variety of ways. As a result, any function disclosed herein as being performed by the transcript generating module 110 may additionally or alternatively be performed by the speech recognition and understanding module 140, and vice versa. The transcript generating module 110 may, for example, use the ASR engine of the speech recognition and understanding module 140 to generate, based on the audio signal 150, text in the transcript 152 representing speech in the audio signal 150.

For example, as described above, the audio signal 150 may include a first audio signal representing the speech of a first person and a second audio signal representing the speech of a second person. The transcript generating module 110 may generate first text representing the speech of the first person and second text representing the speech of the second person. The transcript generating module 110 may, for example, use the speaker change detection module and speaker identification module described above to identify changes of speaker in the audio signal 150 and to identify individual speakers within the audio signal 150 (such as to identify which portions of the audio signal 150 represent speech of a first person (e.g., a physician) and which portions of the audio signal 150 represent speech of a second person (e.g., a patient)). The transcript generating module 110 may perform these functions to generate, in the transcript 152, the first text representing the speech of the first person and the second text representing the speech of the second person. The transcript generating module 110 may also include, within the transcript 152, data representing the identity of the first person and data associating the identity of the first person with the text in the transcript 152 that represents the speech of the first person. Similarly, the transcript generating module 10 may include, within the transcript 152, data representing the identity of the second person and data associating the identity of the second person with the text in the transcript 152 that represents the speech of the second person.

The transcript 152 may include one or more of free-form text, structured text, and discrete data. Free-form text is text that is written in a natural language and that is not accompanied by computer-processable data (e.g., XML tags) that associate a meaning with the text. Structured text is text that is accompanied by computer-processable data (e.g., XML tags) that associate a meaning with the text; structured text includes both such text and the accompanying computer-processable data. Discrete data are data (such as a value of a field in a database table) that have discrete values and which have meanings that are computer-processable. The transcript generating module 110 may, for example, use any of the techniques disclosed in U.S. Pat. No. 7,584,103 B2 (entitled “Automated Extraction of Semantic Content and Generation of a Structured Document From Speech,” issued on Sep. 1, 2009) and U.S. Pat. No. 7,716,040 B2 (entitled, “Verification of Extracted Data,” issued on May 11, 2010) to extract concepts from the audio signal 150 and to generate structured text representing those concepts in the transcript 152. For example, the transcript 152 may include at least: (1) first text representing the speech of the first person and first discrete data representing a first concept represented by the first text; and (2) second text representing the speech of the second person. The transcript may include second discrete data representing a second concept represented by the second text.

Transcript determination module 112 may be configured to determine whether the transcript 152 satisfies a trigger condition associated with a first one of the semantic templates 130a-n (FIG. 2A, operation 206). The first template may include first template text and a first field, as shown within the semantic templates 130a-n in FIG. 1. Determining whether the transcript 152 satisfies the trigger condition may include determining whether the transcript 152 includes content (e.g., free-form text, structured text, and/or discrete data) that satisfies the trigger condition (which may, for example, be a semantic condition). Determining whether the transcript 152 satisfies the trigger condition may include identifying a plurality of templates associated with a plurality of corresponding trigger conditions that are satisfied by the transcript 152.

The plurality of templates that satisfy the trigger condition may include the first template. Determining whether the transcript 152 satisfies the trigger condition may include identifying, from among the plurality of templates, the first template as a best match for the template. For example, the transcript determination module 112 may generate or otherwise identify a distinct match score for each of the plurality of templates that satisfy the trigger condition, and determine that the match score associated with the first template is the best (e.g., highest or lowest) match score among the match scores of the plurality of templates satisfying the trigger condition.

Determining whether the transcript 152 satisfies the trigger condition associated with the first template may include determining whether particular text in the transcript 152 satisfies the trigger condition associated with the first template. The particular text may, for example, be free-form text and/or structured text.

Determining whether the transcript 152 satisfies the trigger condition associated with the first template may include determining whether the transcript 152 and external data (such as data in the external resources 120) satisfy the trigger condition. For example, as described above, determining whether the transcript 152 satisfies the trigger condition associated with the first template may include determining whether the transcript 152 includes particular text, a particular concept, or a particular data element. The external data may be external to the transcript 152 and/or be external to the first template. The external data may include data in an electronic health record that is external to the transcript 152 and that is external to the template. Determining whether the transcript 152 satisfies the trigger condition may take into account a context of the transcript 152, such as a context of the physician-patient dialogue represented by the transcript. As a particular example, if the patient came to the physician for a foot exam related to diabetes, the detection that the patient came to the physician for a foot exam related to diabetes may trigger the selection of a particular content template.

Template insertion module 114 may be configured to, in response to determining that the transcript 152 satisfies the trigger condition associated with the first template, insert the first template into the transcript 152 (FIG. 2A, operation 208). The first template may be one of the content templates 132 of one of the semantic templates 130a-n. As this implies, inserting the first template into the transcript 152 may include inserting text from the first template (e.g., free-form text and/or structured text) into the transcript 152. Any reference herein to inserting a template (e.g., the first template) into the transcript 152 should be understood to include inserting only the textual portion of the template (e.g., the content template 132 and not the corresponding trigger content description 134) into the transcript 152.

Inserting the first template into the transcript 152 may include inserting the first template into the transcript 152 at a location of the particular text in the transcript 152, such as by inserting the immediately before the particular text in the transcript 152 immediately before the particular text in the transcript 152, immediately after the particular text in the transcript 152, or by replacing the particular text in the transcript 152 with the first template. The first template may include data specifying the trigger condition (e.g., the trigger content description 134). The data specifying the trigger condition may specify a semantic description of the trigger condition. The first template may include data specifying an insertion point, i.e., a point at which to insert the first template into the transcript 152. Inserting the first template into the transcript 152 may include inserting the first template into the transcript at the insertion point specified by the first template. The insertion point may specify, for example, a specific section in which to insert the template, a specific location within a section at which to insert the template (e.g., the beginning or the end of the section), or a point relative to other content (e.g., before content representing a specified concept).

The first template may include data indicating whether the first template is repeatable or singular. An example of a repeatable template is an allergy template, in which there may be one allergy template per allergy. An example of a singular template is a smoking cessation template, because only one such template is to be inserted into the transcript 152 no matter how many times the physician mentions the concept of smoking cessation.

The first template may include data indicating one or more post-processing steps to be performed on the first template after the first template has been inserted into the transcript 152. After the template insertion module 114 inserts the first template into the transcript 152, the template insertion module 114 may perform the post-processing step(s) specified by the first template on the first template. Examples of post-processing steps include modifying text in the first template to correct grammatical errors, changing singular to plural (or vice versa), and changing gender pronouns.

As mentioned above, the system 100 may insert content into a template (e.g., into one or more fields of the template) before or after inserting the template into the transcript 152. FIG. 2B illustrates a method 200b that is performed by the system 100 in certain embodiments to perform such content insertion.

Insertion content identifying module 116 may be configured to identify, based on the first template, insertion content to insert into the first template (FIG. 2B, operation 210). Identifying the insertion content may include identifying the insertion content in the transcript 152. Identifying the insertion content may include identifying the insertion content in a source that is external to the transcript 152, such as the external resources 120 (e.g., an EHR). The insertion content may, for example, include text (e.g., free-form text and/or structured text) and/or discrete data. The insertion content may, for example, include data representing an encoded concept, such as structured text representing an encoded concept.

Insertion content insertion module 118 may be configured to insert the insertion content into the first field in the first template (FIG. 2B, operation 212). The insertion content insertion module 118 may insert the insertion content into the first field in the first template before or after the first template has been inserted into the transcript 152.

In some implementations, computing platform(s) 102, remote platform(s) 104, and/or external resources 120 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which computing platform(s) 102, remote platform(s) 104, and/or external resources 120 may be operatively linked via some other communication media.

A given remote platform 104 may include one or more processors configured to execute computer program modules. The computer program modules may be configured to enable an expert or user associated with the given remote platform 104 to interface with system 100 and/or external resources 120, and/or provide other functionality attributed herein to remote platform(s) 104. By way of non-limiting example, a given remote platform 104 and/or a given computing platform 102 may include one or more of a server, a desktop computer, a laptop computer, a handheld computer, a tablet computing platform, a NetBook, a Smartphone, a gaming console, and/or other computing platforms.

External resources 120 may include sources of information outside of system 100, external entities participating with system 100, and/or other resources. In some implementations, some or all of the functionality attributed herein to external resources 120 may be provided by resources included in system 100.

Computing platform(s) 102 may include electronic storage 122, one or more processors 124, and/or other components. Computing platform(s) 102 may include communication lines, or ports to enable the exchange of information with a network and/or other computing platforms. Illustration of computing platform(s) 102 in FIG. 1 is not intended to be limiting. Computing platform(s) 102 may include a plurality of hardware, software, and/or firmware components operating together to provide the functionality attributed herein to computing platform(s) 102. For example, computing platform(s) 102 may be implemented by a cloud of computing platforms operating together as computing platform(s) 102.

Electronic storage 122 may comprise non-transitory storage media that electronically stores information. The electronic storage media of electronic storage 122 may include one or both of system storage that is provided integrally (i.e., substantially non-removable) with computing platform(s) 102 and/or removable storage that is removably connectable to computing platform(s) 102 via, for example, a port (e.g., a USB port, a firewire port, etc.) or a drive (e.g., a disk drive, etc.). Electronic storage 122 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 122 may include one or more virtual storage resources (e.g., cloud storage, a virtual private network, and/or other virtual storage resources). Electronic storage 122 may store software algorithms, information determined by processor(s) 124, information received from computing platform(s) 102, information received from remote platform(s) 104, and/or other information that enables computing platform(s) 102 to function as described herein.

Processor(s) 124 may be configured to provide information processing capabilities in computing platform(s) 102. As such, processor(s) 124 may include one or more of a digital processor, an analog processor, a digital circuit designed to process information, an analog circuit designed to process information, a state machine, and/or other mechanisms for electronically processing information. Although processor(s) 124 is shown in FIG. 1 as a single entity, this is for illustrative purposes only. In some implementations, processor(s) 124 may include a plurality of processing units. These processing units may be physically located within the same device, or processor(s) 124 may represent processing functionality of a plurality of devices operating in coordination. Processor(s) 124 may be configured to execute modules 108, 110, 112, 114, 116, and/or 118, and/or other modules. Processor(s) 124 may be configured to execute modules 108, 110, 112, 114, 116, and/or 118, and/or other modules by software; hardware; firmware; some combination of software, hardware, and/or firmware; and/or other mechanisms for configuring processing capabilities on processor(s) 124. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 108, 110, 112, 114, 116, and/or 118 are illustrated in FIG. 1 as being implemented within a single processing unit, in implementations in which processor(s) 124 includes multiple processing units, one or more of modules 108, 110, 112, 114, 116, and/or 118 may be implemented remotely from the other modules. The description of the functionality provided by the different modules 108, 110, 112, 114, 116, and/or 118 described below is for illustrative purposes, and is not intended to be limiting, as any of modules 108, 110, 112, 114, 116, and/or 118 may provide more or less functionality than is described. For example, one or more of modules 108, 110, 112, 114, 116, and/or 118 may be eliminated, and some or all of its functionality may be provided by other ones of modules 108, 110, 112, 114, 116, and/or 118. As another example, processor(s) 124 may be configured to execute one or more additional modules that may perform some or all of the functionality attributed below to one of modules 108, 110, 112, 114, 116, and/or 118.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer (e.g., the computing platform(s) 102) including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention apply automatic speech recognition and natural language processing to automatically (i.e., without human intervention) generate speech from text. Such functions are inherently computer-implemented.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

What is claimed is:

1. A method performed by at least one computer processor executing computer program instructions stored on at least one non-transitory computer-readable medium, the method comprising:
receiving an audio signal, the audio signal representing a dialog between a first person and a second person, the audio signal including a first audio signal representing speech of the first person and a second audio signal representing speech of the second person;
generating, based on the first audio signal and the second audio signal, a transcript of the dialog, the transcript comprising:
first text representing the speech of the first person;
first discrete data representing a first concept represented by the first text;
second text representing the speech of the second person; and
second discrete data representing a second concept represented by the second text;
determining whether the transcript satisfies a trigger condition associated with a first template; and
in response to determining that the transcript satisfies the trigger condition associated with the first template, inserting the first template into the transcript.

2. The method of claim 1, wherein the first template comprises first template text and a first field.

3. The method of claim 2, further comprising:
identifying, based on the first template, insertion content to insert into the first template; and
inserting the insertion content into the first field in the first template.

4. The method of claim 3, wherein identifying the insertion content comprises identifying the insertion content in the transcript.

5. The method of claim 3, wherein identifying the insertion content comprises identifying the insertion content in a source that is external to the transcript.

6. The method of claim 3, wherein the insertion content comprises text.

7. The method of claim 3, wherein the insertion content comprises an encoded concept.

8. The method of claim 1, wherein determining whether the transcript satisfies the trigger condition comprises determining whether the transcript includes content that satisfies the trigger condition.

9. The method of claim 8, wherein determining whether the transcript includes content that satisfies the trigger condition comprises determining whether the transcript includes content that satisfies a semantic condition specified by the trigger condition.

10. The method of claim 1, wherein determining whether the transcript satisfies the trigger condition comprises:
identifying a plurality of templates associated with a plurality of corresponding trigger conditions that are satisfied by the transcript, the plurality of templates including the first template; and
identifying, from among the plurality of templates, the first template as a best match for the template.

11. The method of claim 1, wherein determining whether the transcript satisfies the trigger condition associated with the first template comprises determining whether particular text in the transcript satisfies the trigger condition associated with the first template, and
wherein inserting the first template into the transcript comprises inserting the first template into the transcript at a location of the particular text in the transcript.

12. The method of claim 1, wherein the first template comprises data specifying the trigger condition.

13. The method of claim 12, wherein the data specifying the trigger condition specifies a semantic description.

14. The method of claim 1, wherein determining whether the transcript satisfies the trigger condition associated with the first template comprises determining whether the transcript and external data satisfy the trigger condition, wherein the external data are external to the transcript and wherein the external data are external to the first template.

15. The method of claim 14, wherein the external data comprise data in an Electronic Health Record (EHR) that is external to the transcript and that is external to the template.

16. A system comprising at least one non-transitory computer-readable medium having computer program instructions stored thereon, the computer program instructions being executable by at least one computer processor to perform a method, the method comprising:
receiving an audio signal, the audio signal representing a dialog between a first person and a second person, the audio signal including a first audio signal representing speech of the first person and a second audio signal representing speech of the second person;
generating, based on the first audio signal and the second audio signal, a transcript of the dialog, the transcript comprising:
first text representing the speech of the first person;
first discrete data representing a first concept represented by the first text;
second text representing the speech of the second person; and
second discrete data representing a second concept represented by the second text;
determining whether the transcript satisfies a trigger condition associated with a first template; and
in response to determining that the transcript satisfies the trigger condition associated with the first template, inserting the first template into the transcript.

17. The system of claim 1, wherein the first template comprises first template text and a first field.

18. The system of claim 17, wherein the method further comprises:
identifying, based on the first template, insertion content to insert into the first template; and
inserting the insertion content into the first field in the first template.

19. The system of claim 18, wherein the insertion content comprises an encoded concept.

20. The system of claim 16, wherein determining whether the transcript satisfies the trigger condition comprises determining whether the transcript includes content that satisfies the trigger condition.
